(19) United States
(12) Patent Application Publication
(10) Pub. No.: US 2019/0167379 A1
(43) Pub. Date: Jun. 6, 2019

(54) POSITIONING MARKER

(71) Applicant: Ingemar Näslund, Huddinge (SE)

(72) Inventor: Ingemar Näslund, Huddinge (SE)

(21) Appl. No.: 15/846,810

(22) Filed: Dec. 19, 2017

(30) Foreign Application Priority Data
Dec. 19, 2016 (SE) 1651672-6

(51) Int. Cl. A61B 90/00

(52) U.S. Cl. CPC A61B 90/39; A61B 2090/3995; A61B 2090/3991; A61B 2090/3987

(57) ABSTRACT

The present invention relates to an implantation assembly comprising a hollow needle (20) with a longitudinal axis (L) and an opening slanted at an first acute angle in relation to a plane perpendicular to said longitudinal axis (L), a mandrel (21) adapted to slide within the inside the needle along the longitudinal axis (L), and a plurality of positioning markers (10), wherein each of the plurality of markers (10) comprises an elongated marker body (11) with a proximal end (12) and a distal end (13), wherein said proximal end (12) and said distal end (13) of said marker body (11) is slanted essentially at a same second acute angle (α) in relation to the plane, wherein said plurality of positioning markers (10) are mounted distally of said mandrel (21) within said needle (20) and proximally of the distal end of said needle (20) before implantation, and said plurality of positioning markers (10) being adapted to be implanted using said hollow needle (20) and said mandrel (21), wherein the marker body (11) of each of the said plurality of positioning markers (10) is adapted to tilt and rotate in reference to the longitudinal axis (L) of the needle after exiting the slanted opening of the needle (20).

### FIELD OF THE INVENTION

The present invention relates to a positioning marker that is intended to be inserted in tissue according to the preamble of the independent claim. The invention further relates to an implantation assembly for inserting a plurality of positioning markers.

### BACKGROUND OF THE INVENTION

When treating cancer tumors the physician commonly prepares the treatment e.g. by planning how large the dose of the medicine shall be or how much radiation and what part of the tissue should be subjected to radiation therapy. Basis for the planning of the radiation therapy is done e.g. by using computer tomography images. Radiotherapy clinics and hospitals use MRI (magnetic resonance imaging) systems in order to improve image quality for target plotting, providing them with new possibilities to develop tailored cancer treatments and follow-ups of treatment results. Plotted tumor areas are transferred to CT (computer tomography) images which are used as a basis for dose planning. Registering the images is made easier and safer by using markers that are visible on all the above types of images.

In order to minimize the side effects on normal healthy tissue repeatedly radiation doses normally are given. At these times of treatment it is important to carefully reposition the body and the tumor area according to predetermined parameters so the tumor area not will be missed, and healthy tissue radiation is minimized.

In radiotherapy positioning is commonly carried out using positioning markers, also called fiducial markers. The use of markers in online positioning reduces both systematic and random errors and generally provides high quality therapy, as using fiducial markers in the positioning procedure leads to the possibility of using of very narrow margins around the tumor. This substantially reduces undesirable adverse effects in healthy tissue and concentrates the dose to the actual tumor.

The fiducial markers are commonly implanted with needles. Most markers on the market, whether made of gold, carbon, ceramic or other materials require needles that have diameters of about 1.25 mm to 1.50 mm (18 to 17G). A few more recent markers are smaller in size, with needle diameters of 0.50 mm or 0.70 mm (25G or 22G), such as described in WO2012/154116.

US 2014/0276037 shows a fiducial marker of a cylindrical shape, used in a needle assembly adapted to eject the marker out of a side opening in the needle. Another example of a marker is shown in US2005/0143650, which discloses an elongated marker with is adapted to form a bent shape when implanted, such that it stays in place in the tissue.

US 2016/0074131 disclose a marker with a generally cylindrical shape and provided with threads adapted to match threads on a loading needle, in order to deploy one or several markers in a rotational manner.

Positioning markers that are placed into tissue of human or animal will rest mainly in the same place for the entire life of the subject. Thus, it is extremely important that the markers not cause mechanical damage or give rise to allergies or other state of ill-health.

The marker must further have sufficient size and have an appropriate density in order for the marker to be clearly depicted in at least one type, preferably several types, of imaging techniques.

A marker's outer diameter commonly corresponds to the needle's inner diameter and will, with most marker types, after implantation, be slightly smaller than the opening caused by the implantation needle. Therefore, a problem with existing solutions is that many of the larger conventional markers are, after placement in tissue, able to move much like a piston in a cylinder as e.g. blood presses against the marker.

Furthermore, local anesthesia, necessary for implantation using larger needles, has an effect on the general anatomy, and patients are therefore usually sent home 1 to 2 weeks after implantation to give the markers time to stabilize and the tissue to normalize before continuing with CT-MRI treatment for dose planning. This lead-time can be reduced or completely omitted by using a smaller diameter marker which expands to a slightly larger size than the needle diameter after implantation, by e.g. forming an entangled structure, thus preventing the marker from being able to move after implantation. Foldable/expandable markers are especially suited for systems with automatic marker detection. The small initial diameter enables use of a smaller needle, making local anesthesia unnecessary in most cases. However, such a foldable marker is more expensive to manufacture.

The inventor of the present invention has identified a need for an improved positioning marker, which provides for improved stability in placement in tissue over time and which is less expensive to manufacture than commonly known markers.

### SUMMARY OF THE INVENTION

An object of the present invention is to provide a positioning marker which stays in place in tissue for an extended period of time.

A further object is to provide a positioning marker which can be used with small gauge needles, to minimize tissue trauma.

Yet another object is to provide a positioning marker which is cost effective to manufacture and suitable for use with commonly used needles.

The above-mentioned objects are achieved by the present invention according to the independent claim. Preferred embodiments are set forth in the dependent claims.

In accordance with the present invention the positioning marker comprises a positioning marker intended to be implanted into tissue, wherein the marker is an elongated object with a longitudinal axis and with a diameter perpendicular to the longitudinal axis. The marker is intended to be implanted using a hollow needle and a mandrel. The marker comprises a marker body with a proximal end and a distal end, which is adapted to be arranged in the needle such that the longitudinal axis of the marker body corresponds essentially to the longitudinal axis of the needle before implantation. The proximal end of the marker body is slanted at an angle in relation to a plane perpendicular to the longitudinal axis of the marker.

In accordance with the present invention, the implantation assembly comprises a hollow needle with a longitudinal axis and an opening slanted at an first acute angle in relation to a plane perpendicular to said longitudinal axis, a mandrel adapted to slide within the inside the needle along the longitudinal axis, and a plurality of positioning markers, wherein each of the plurality of markers comprises an elongated marker body with a proximal end and a distal end, wherein said proximal end and said distal end of said marker body is slanted essentially at a same second acute angle in relation to the plane, wherein said plurality of positioning markers are mounted distally of said mandrel within said needle and proximally of the distal end of said needle before implantation, and said plurality of positioning markers being adapted to be implanted using said hollow needle and said mandrel, wherein the marker body of each of the said plurality of positioning markers is adapted to tilt and rotate in reference to the longitudinal axis of the needle after exiting the slanted opening of the needle.

### SHORT DESCRIPTION OF THE APPENDED DRAWINGS

FIGS. 1a-1d illustrate positioning markers according to prior art.

FIG. 2 illustrate a positioning marker in use as disclosed herein.

FIGS. 3a-3c show examples of the shape of a positioning marker.

FIGS. 4a-4c illustrates the steps of placement of a positioning marker in tissue.

FIGS. 5a-5d illustrates the steps of placement of several sequential positioning markers in tissue.

### DETAILED DESCRIPTION

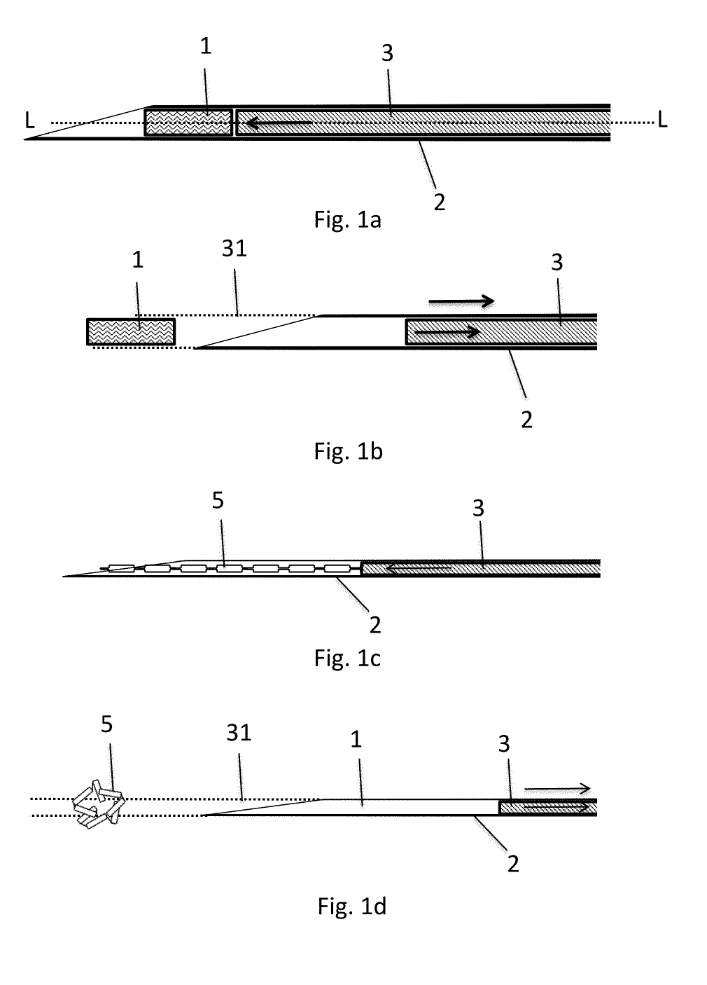

FIGS. 1a-d shows two types of commonly used conventional positioning markers used in radiotherapy, and how the marker is placed in tissue. Notably, in these and the following figures, if not specifically indicated otherwise, the left sides of the figures and the depicted devices represent the distal direction, i.e. the direction of deeper penetration into tissue, and away from the user. Thus, the right sides of the figures correspondingly show a position more proximal to a user, or closer to the skin or outside of the body, when the needle is placed into tissue. It is to be understood that the needles shown are only depicted at their distal end, and may be used to penetrate into tissue at any desired depth, i.e. a marker may be placed closed to the tissue surface, e.g. just under the skin, or deep into tissue in a body. Commonly the mandrel and/or needle may have a handle at the very proximal end (not shown), for manipulation of the relative position of the mandrel in relation to the hollow needle, for pushing a marker in a distal direction and out of the needle when placing the marker into tissue. After expulsion of the marker from the needle, the needle and mandrel are pulled back out of the tissue and body.

FIGS. 1a and 1b illustrate the position of a, typically cylindrical, marker 1 before and after, respectively, placement of a marker as known in the art. The marker 1 is placed by being pushed out of a hollow needle 2 using a mandrel 3, and ends up in the tissue channel 31 created by the needle, essentially along the longitudinal axis L of the needle 2. To be visible, such a marker must be of a relatively large diameter, and consequently requires a relatively large diameter needle. Thus, the channel 31 created in the tissue during the procedure will be quite large, allowing the placed marker to move freely in the channel, much like a piston in a chamber. This creates uncertainty in the positioning function of the marker, as it might, or might not, move between, or even during, radiotherapy sessions.

FIGS. 1c and 1d illustrate another type of marker as known in the art, and show the position of a foldable marker 5 before and after, respectively, placement in tissue. Such a marker 5 may, as shown, comprise multiple segments of a wire-like structure, which, when pushed out of a needle 2, folds or crumples into a three-dimensional structure that has a larger diameter than the needle, or at least a larger diameter than the initial diameter of the marker 5. Such a marker has the advantage of lodging itself securely into the tissue, as it will have a larger diameter than the tissue channel 31 when folded, thus minimizing the risk of movement over time. Furthermore, such a marker may be of considerably smaller diameter when unfolded, and still be clearly visible in an MRI, X-ray or CT images when in a folded state. Therefore, a foldable marker may thus be placed using a considerably smaller diameter needle, which minimizes trauma to the patient, and may be performed even without general anesthesia. The time between marker placement and radiotherapy may consequently be lessened of even omitted.

In the present disclosure, an improved fiducial marker for implantation into mammalian tissue using a needle and mandrel is shown. One such marker is shown in FIG. 3a, which is a side view of a positioning marker 10. The marker 10 is an elongated object, preferably with a circular cross-section, and with a longitudinal axis A and with a diameter d perpendicular to said longitudinal axis, and wherein the marker is intended to be implanted using a hollow needle and a mandrel as will be described further below.

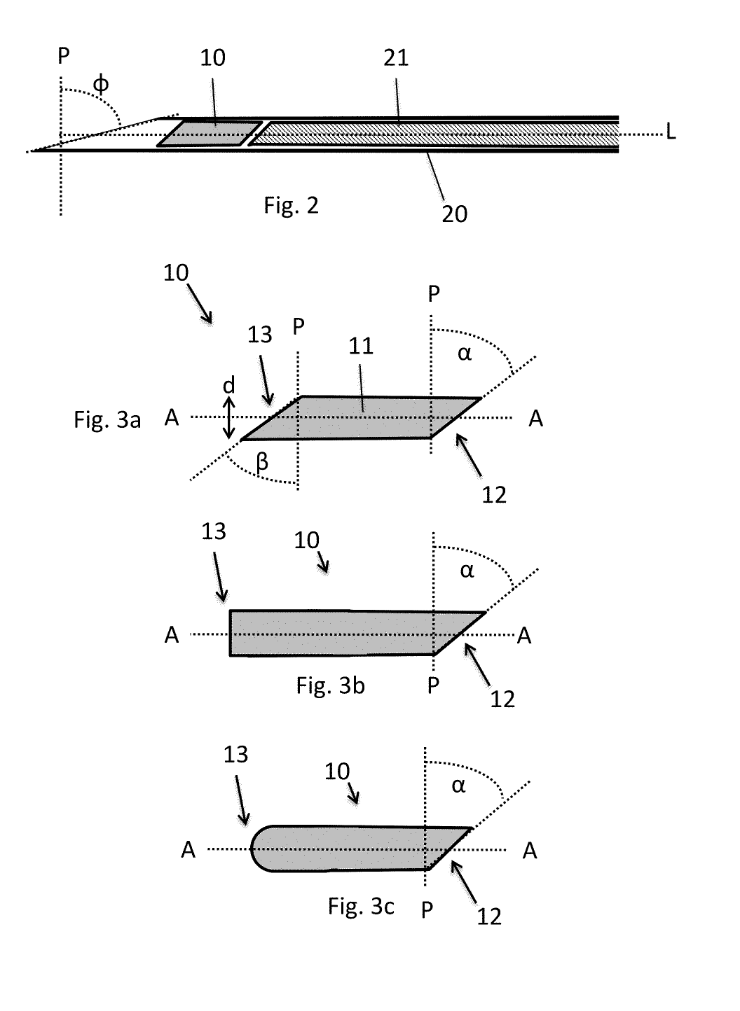

The positioning marker 10 comprises a marker body 11 with a proximal end 12 and a distal end 13. As is understood from the above, the distal end is intended to be the end to first exit the needle and enter the tissue when the marker is ejected from the distal end of the needle. The marker body is adapted to be mounted in a hollow needle 20 such that the longitudinal axis A of the marker body 11 corresponds essentially to the longitudinal axis L of the needle 20 before implantation. This is shown in FIG. 2.

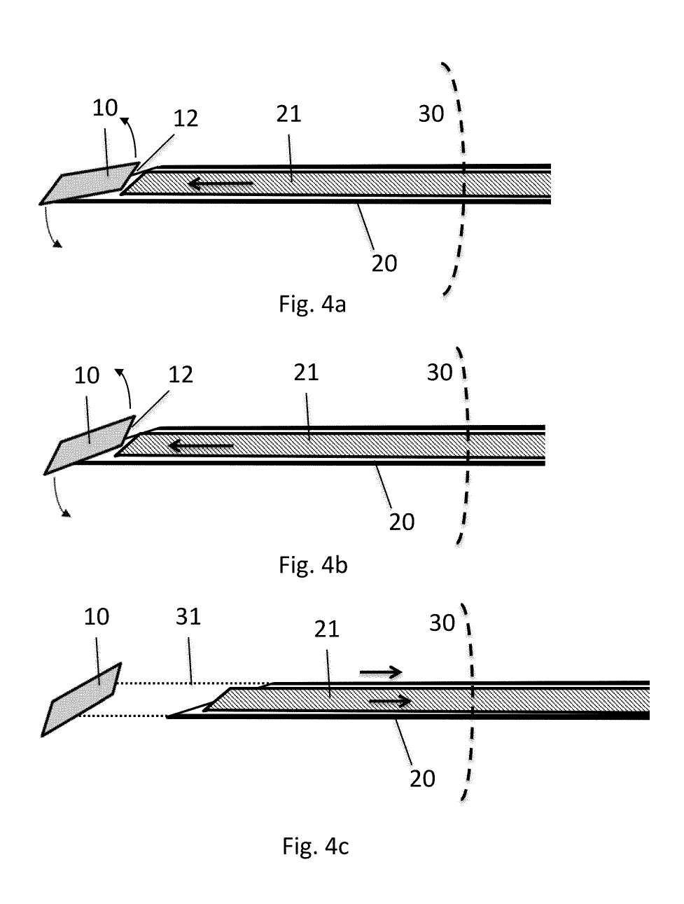

The proximal end 12 of the marker body is slanted at an angle α in relation to a plane P perpendicular to the longitudinal axis A of the marker body 11. Such a slanted surface at the proximal end 12 of the marker 10 will cause tilting when it is pushed out of or exits the needle or needle opening. To understand this, the procedure of implanting the marker is illustrated in FIGS. 4a-4c, showing three sequential steps in the implantation procedure. The dashed line in FIGS. 4a-4c illustrates a fictive tissue edge or border, wherein the marker is intended to be implanted into, depending on the radiotherapy application. However, as mentioned above, the implantation depth into tissue is not relevant to the following implantation steps, and a marker may be implanted at any desired depth into a tissue. Initially, the marker 10 is placed in a hollow needle 20, distally of a suitable mandrel 21 for pushing the marker distally in the needle. An example of an initial configuration is shown in FIG. 2.

The needle 20 and mandrel 21 may have any commonly known dimensions, as long as the mandrel 21 is adapted to be maneuvered inside the needle 20, e.g. distally inside the lumen of the needle 20, and lengths of the needle 20 and mandrel 21 are chosen such that the distal tip of the mandrel 21 may be pushed out beyond the distal end of the hollow needle 20, as will become apparent below. The mandrel 21 and/or needle 20 may each have a proximal handle at the proximal end (not shown), for manipulation of the relative position of the mandrel 21 in relation to the hollow needle 20 along a longitudinal axis, such that the mandrel 21 is adapted for pushing a marker in a distal direction and out of the needle 20 when placing the marker into tissue. Preferably, the mandrel may have any shape at its distal end. In FIGS. 2, 4 and 5 the distal end of the mandrel 21 is adapted to correspond to the slanted surface of the proximal end 12 of the marker 10. However, the distal end of the mandrel may have other shapes, as will be further described below.

During an implantation procedure, the assembly or implantation assembly comprising a needle 20, mandrel 21 and marker 10 is initially in the configuration shown in FIG. 2. The marker 10 is located inside the distal end of the needle 20. In this configuration, the needle 20 is inserted into the desired tissue 30 until the tip of the assembly is in essentially the location where the marker is to be implanted. As seen in FIG. 4a, the mandrel 21 is then manipulated in a distal direction, as illustrated by the straight arrow, such that it pushes the marker 10 out of the distal end of the needle 20.

In one embodiment, the implantation assembly comprises a hollow needle 20 with a longitudinal axis L and an opening slanted at an first acute angle in relation to a plane perpendicular to said longitudinal axis L. In embodiments, the implantation assembly further comprises a mandrel 21 adapted to slide within the inside the needle along the longitudinal axis L. In embodiments, the implantation assembly further comprises a plurality of positioning markers 10, wherein each of the plurality of markers 10 comprises an elongated marker body 11 with a proximal end 12 and a distal end 13, wherein said proximal end 12 and said distal end 13 of said marker body 11 are slanted essentially at a same second acute angle (α) in relation to the plane. In embodiments, the plurality of positioning markers 10 are mounted distally of said mandrel 21 within said needle 20 and proximally of the distal end of said needle 20 before implantation, and said plurality of positioning markers 10 are adapted to be implanted using said hollow needle 20 and said mandrel 21. In embodiments, the marker body 11 of each of the said plurality of positioning markers 10 is adapted to tilt and rotate in reference to the longitudinal axis L of the needle after exiting the slanted opening of the needle 20.

As shown in FIG. 4a, and further in FIG. 4b, when the mandrel 21 is pushed forward, i.e. distally, and out of the needle 20, the mandrel 21 pressing on the slanted proximal end 12 of the marker 10 will cause the marker to immediately start tilting (rotating). This is shown by the curved arrows. Notably, the inclination of the marker 10 to tilt or rotate due to the force applied on the slanted proximal end 12 is present already inside the needle, but while the marker 10 is inside the needle 20, it is restricted in movement by the walls of the needle, and can thus only move along the longitudinal axis L of the needle.

When the mandrel 21 continues to press against the slanted proximal end of marker 10, on exiting the distal end of the needle 20, the marker will tilt and lodge itself in a position partly out of line with the insertion direction, and with the longitudinal axis L of the needle. As shown in FIG. 4c, this causes the marker 10 to tightly lodge itself in the tissue 30, and minimizes the risk of the marker moving (e.g. like a piston) in the channel 31 formed by the insertion needle. The needle and mandrel are pulled out of the tissue after implantation.

In addition to being angled or slanted at the proximal end 12, a marker may also be slanted at the distal end 13. Such a marker is preferably slanted at the distal end at an angle β in relation to a plane P perpendicular to the longitudinal axis A of the marker body 11. One such example is shown in FIG. 3a. Preferably, the angle β provided at the distal end 13 is essentially the same as the angle α provided at the proximal end 12. Such an arrangement is especially advantageous from a manufacturing standpoint, as multiple markers may be made by cutting a cylindrical rod of material at a specified angle along its length.

In an embodiment, the angle α provided at the proximal end 12 may in one embodiment be substantially or essentially the same as the angle β provided at the distal end 13, The angle α may be selected in the range [30 degrees to 70 degrees], more preferably in the range [40 degrees to 50 degrees] and most preferably selected as 45 degrees.

As an example, the two slanted ends may be provided essentially parallel to each other, as indicated in FIG. 3a. This provides for a manufacturing method wherein a cylindrical rod of material may be cut at the same angle at equidistant points along the rod. Further, a marker 10 with the two slanted ends provided essentially parallel to each other will improve the rotational effect when exiting the needle.

Providing a slanted or angled distal end, in addition to a slanted proximal end, may enhance tilting function of a marker, in that the front (distal) end of a marker is pointed and will lodge itself into tissue as soon as the marker exits the insertion needle, as seen in e.g. FIG. 4a-4c, and enhance the tilting initiated by the mandrel pressing against the slanted proximal end of the marker.

As an alternative, a marker 10 may potentially also be flat or rounded at the distal end, as shown in FIGS. 3b and 3c. These types of markers will tilt as described above, due to the slanted proximal end, as illustrated in e.g. FIG. 4a-4c.

A marker 10 has a predetermined total length (L_{TOT}), which is preferably in the range of 1 mm to 10 mm, more preferably in the range of 2 mm to 6 mm.

The marker may be used with any known type of mandrel for implantation, as long as the mandrel used is adapted to the used needle. The mandrel may be slanted in the distal end, as shown in the figures, but may also be flat, rounded or (symmetrically) tapered, or pointed.

The mandrel may be slanted at an angle in relation to a plane perpendicular to the longitudinal axis L of needle 20. The slanted angle of the mandrels distal end may in one embodiment be substantially or essentially the same as the angle α and/or the angle β provided of the marker. The slanted angle may be selected in the range [30 degrees to 70 degrees], more preferably in the range [40 degrees to 50 degrees] and most preferably selected as 45 degrees.

The diameter d of the marker may be any diameter suitable for commonly used needles, such as in the range of 0.28 mm to 2.0 mm, more preferably in the range of 0.28 mm to 0.7 mm and most preferably in the range of 0.28 mm to 0.4 mm. The shape is preferably generally rod-shaped, but can also have any other suitable cross-sectional shape, such as square or triangular. The latter might require a specially adapted implantation needle.

The marker is preferably made of a material visible in several different types of imaging techniques, such as CT, MRI, X-Ray etc. One example is an alloy or granulation mixture of gold with small amount of a ferromagnetic or paramagnetic material, e.g. iron.

As mentioned above, a marker according to the present disclosure is provided with a proximal end 12 of the marker body 11 which is slanted at an angle α in relation to a plane P perpendicular to the longitudinal axis A of the marker body 11. The angle α is preferably in the range of 30 to 70 degrees, more preferably 45 to 60 degrees.

In a marker 10 wherein also the distal end 13 may be provided with a slanted end, the angle β in relation to a plane P perpendicular to the longitudinal axis A of the marker body 11 is preferably in the range of 0 to 70 degrees, more preferably 45 to 60 degrees. As mentioned, angle β, at the distal end 13, may be essentially the same angle as angle α, at the proximal end 12.

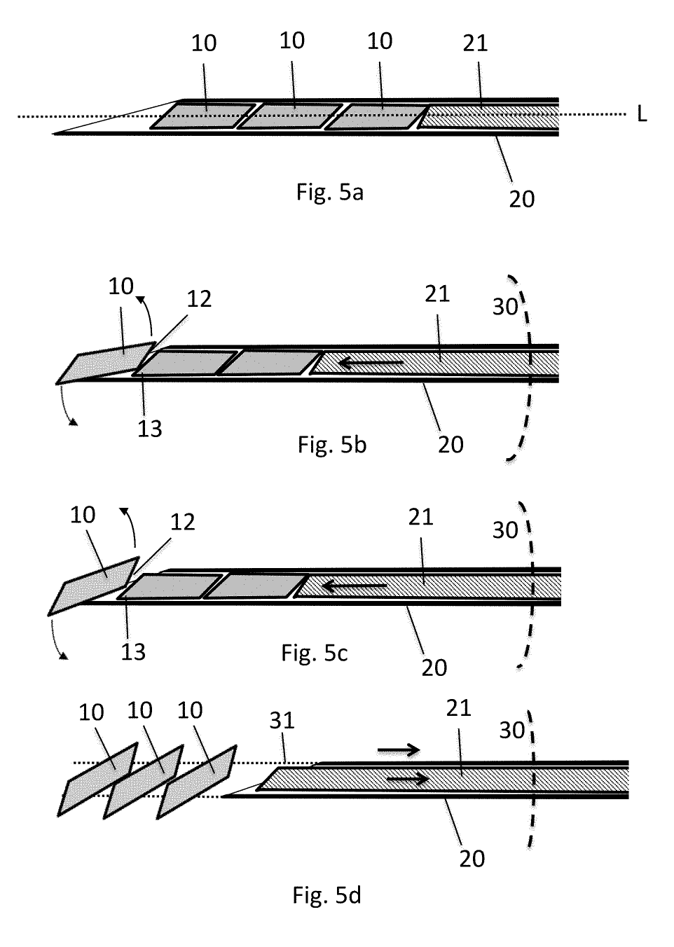

The markers disclosed above may also be adapted for implantation of two or more markers, i.e. a plurality of markers in a similar manner. As illustrated in FIGS. 5a to 5d, several markers 10 may be mounted adjacent to each other and in a row in a hollow needle. The markers are arranged in the needle 20 distally of a suitable mandrel 21, as previously described. Preferably, the markers 10 used are essentially identical in shape, and have both a slanted proximal end 12 and a slanted distal end 13, which ends preferably are essentially parallel to each other, as described above, and shown in detail in FIG. 3a. Such an arrangement provides for advantages such as both ease of manufacture of the markers 10, as described above, and also provide for enhanced functionality, in that the markers 10 may initially be arranged tightly stacked against each other in the needle 20, as shown in FIG. 5a. This figure shows the arrangement of an assembly comprising two or more markers before implantation and during insertion into the desired position in the tissue. In this initial configuration all the markers 10 are arranged such that their longitudinal axes A are essentially arranged along the longitudinal axis L of the needle 20. Thus, the assembly comprises a needle 20, mandrel 21 and two or more markers 10 initially in the configuration shown in FIG. 5a. FIGS. 5a-5d show three markers, however the number of markers 10 may be any number from 1 to 15, more preferably 2 to 6.

The insertion procedure for inserting several sequential markers 10 corresponds to that described in connection with FIG. 4a-4c, with the exception that all but the last marker in the sequence is pushed forwards, and tilted (rotated) on exiting the distal end of the needle due to the pressure from the marker behind it, instead of the direct pressure from the mandrel. Thus the pressure from the mandrel 21 is transferred distally through the stack of markers 10 in the needle 20.

Accordingly, as shown in FIG. 5a, the markers 10 are initially located inside the distal end of the needle 20. In this configuration, the needle 20 is inserted into the desired tissue 30 until the tip of the assembly is in essentially the location where the marker is to be implanted. As seen in FIG. 5b, the mandrel 21 is then manipulated in a distal direction, as illustrated by the arrow, such that it pushes the markers 10 distally and out of the distal end of the needle 20 one by one.

As shown in FIG. 5b, and further in FIG. 5c, when the mandrel 21 is pushed forward, i.e. distally, and out of the needle 20, the mandrel 21 presses on the most proximal marker 10 and will cause all the markers 10 to move distally. Due to the slanted proximal end of each marker 10, when each marker exits the distal end it will immediately start tilting, i.e. rotating. This is shown by the curved arrows, and is due to either the distal end 13 of an adjacent marker or the distal tip of the mandrel exerting force on the slanted surface of the proximal end of the marker 10. In other words, as soon as each marker 10 is no longer restricted by the walls of the needle, the pressure on its slanted proximal end 12 will cause the marker to tilt and rotate in reference to the longitudinal axis L of the needle and thus also the tissue channel 31. When the mandrel 21 continues to press against markers 10, each marker 10 will exit the needle 20 and tilt. In other words, a plurality of positioning markers will exit the needle 20 subsequently and thereby grouping together to form a larger body, larger than a single marker, comprised by the plurality of positioning markers. The larger body will improve visibility on all the previously mentioned types of images, e.g. MRI and CT images.

In one embodiment, the markers 10 have a circular, oval, square or triangular cross-sectional shape, seen in a perpendicular cross-section to the longitudinal axis L. In this example, after a first marker has lodged itself in the tissue 30, a subsequent marker will be guided along the side of the first marker and be aligned tightly with and essentially parallel to the first marker once it comes to its resting position, as shown in FIG. 5d.

Consequently, on exiting the distal end of the needle 20, each marker will tilt and lodge itself in a position partly out of line with the insertion direction, and the longitudinal axis L of the needle. As shown in FIG. 5d, this causes the markers 10 to tightly lodge themselves in the tissue 30. Furthermore, the markers 10 will end up essentially parallel to each other, but tilted in relation to the channel 31 formed by the insertion needle. As before, this will minimize the risk of the markers moving (e.g. like a piston) in the channel 31 formed by the insertion needle. Furthermore, when inserting several or a plurality of markers after the other, the markers will collectively provide better visibility of the markers on a suitable imaging system. This is due to using multiple markers instead of a single marker, which then form a larger volume compared to single marker. The needle and mandrel can then be pulled out of the tissue after implantation with the markers secured in the tissue, thus minimizing the risk that one or more markers will move along the tissue channel 31 over time.

Notably, the markers 10 in FIGS. 5a to 5d are illustrated with slanted distal (front) ends 13. As mentioned above, to facilitate manufacturing, and also to enhance the tilting function, both the proximal end 12 and the distal end 13 may be provided with a slanted surface, preferably with essentially parallel surfaces, i.e. with corresponding angles.

The slanting of the distal end of the marker body may further be substantially rotationally aligned around the longitudinal axis L with the slanting of the opening. This will allow a distal end 13 of an adjacent marker or the distal tip of the mandrel to exert force on the slanted surface of the proximal end of the marker 10 even before the marker exits the needle entirely, as can be seen e.g. in FIG. 5b. The slanting of the distal end of the mandrel may further also be substantially rotationally aligned around the longitudinal axis L with the slanting of the opening.

In one example, the slanting of the opening, the slanting of the distal and proximal end of the marker and the slanting of the distal tip of the mandrel have the same angle in relation to a plane perpendicular to the longitudinal axis L. The slanting of the opening, the slanting of the distal and proximal end of the marker and the slanting of the distal tip of the mandrel are then rotationally aligned around the longitudinal axis L such that the slanted surfaces formed by the slanting of the opening, the slanting of the distal and proximal end of the marker and the slanting of the distal tip of the mandrel are substantially arranged in parallel planes.

However, it is also conceivable that markers, either when used individually, as e.g. shown in FIGS. 4a to 4c, or when using multiple markers as shown in FIGS. 5a to 5d, are provided with another shape at the distal end 13, such as a flat end, a rounded or (symmetrically) tapered shape, or a pointed shape.

An implantation assembly may comprise a hollow needle 20, a mandrel 21 adapted to slide within the hollow needle 20, and at least one positioning marker according to any of the above disclosed examples. The marker 10 is initially arranged inside the distal end of the needle 20 and distally of the mandrel within the needle. Such an assembly may be used as described above.

The present invention is not limited to the above-described preferred embodiments. Various alternatives, modifications and equivalents may be used. Therefore, the above embodiments should not be taken as limiting the scope of the invention, which is defined by the appending claims.

1. An implantation assembly comprising:
a hollow needle with a longitudinal axis and an opening slanted at an first acute angle in relation to a plane perpendicular to said longitudinal axis,
a mandrel adapted to slide within the inside the needle along the longitudinal axis, and
a plurality of positioning markers, wherein each of the plurality of markers comprises an elongated marker body with a proximal end and a distal end, wherein said proximal end and said distal end of said marker body is slanted essentially at a same second acute angle in relation to the plane,
wherein said plurality of positioning markers are mounted distally of said mandrel within said needle and proximally of the distal end of said needle before implantation, and said plurality of positioning markers being adapted to be implanted using said hollow needle and said mandrel, wherein the marker body of each of the said plurality of positioning markers is adapted to tilt and rotate in reference to the longitudinal axis of the needle after exiting the slanted opening of the needle after implantation.

2. The implantation assembly according to claim 1, wherein the plurality of positioning markers comprise between 1 and 15 markers, said plurality of positioning markers being mounted sequentially within said needle.

3. The implantation assembly according to claim 1,
wherein the marker body of each of the said plurality of positioning markers is adapted to be arranged in said needle such that a longitudinal axis of said marker body corresponds essentially to the longitudinal axis of said needle before implantation.

4. The implantation assembly according to claim 1, wherein the positioning markers have a diameter perpendicular to said longitudinal axis in the range of 0.28 mm to 2.0 mm, more preferably in the range of 0.28 mm to 0.7 mm and most preferably in the range of 0.28 mm to 0.4 mm.

5. The implantation assembly according to claim 1, wherein each of the markers has a predetermined total length, said predetermined total length being in the range of 1 mm to 10 mm.

6. The implantation assembly according to claim 1, wherein said angle is in the range of 30 to 70 degrees, more preferably in the range of 40 degrees to 50 degrees and most preferably selected as 45 degrees.

7. The implantation assembly according to claim 1, wherein the distal end of the mandrel is slanted at an angle in relation to the plane essentially the same as the second acute angle.

8. The implantation assembly according to claim 7, wherein the slanting of the distal end of the mandrel is substantially rotationally aligned around the longitudinal axis with the slanting of the opening of the needle.

9. The implantation assembly according to claim 1, wherein the slanting of the distal end of the marker body is substantially rotationally aligned around the longitudinal axis with the slanting of the opening of the needle.

10. The implantation assembly according to claim 1, wherein the positioning markers are intended to be implanted into tissue.
